(19) United States
(12) Patent Application Publication
(10) Pub. No.: US 2016/0162633 A1
(43) Pub. Date: Jun. 9, 2016

(54) METHOD OF SCREENING PROTEIN CAPABLE OF BINDING TO COMPOUND

(71) Applicant: Samsung Electronics Co., Ltd., Suwon-si (KR)

(72) Inventors: So Jeong Yun, Hwaseong-si (KR); Young Wan Ha, Seoul (KR); Chihyun Park, Seoul (KR)

(21) Appl. No.: 14/958,282

(22) Filed: Dec. 3, 2015

(30) Foreign Application Priority Data
Dec. 3, 2014 (KR) 10-2014-0172375

(51) Int. Cl. G06F 19/18; G01N 33/68

(52) U.S. Cl. CPC G06F 19/18; G01N 33/6845

(57) ABSTRACT

Provided is a method of screening a protein capable of binding to a specific compound using gene ontology terms.

### RELATED APPLICATION

This application claims the benefit of Korean Patent Application No. 10-2014-172375, filed on Dec. 3, 2014, in the Korean Intellectual Property Office, the entire disclosure of which is hereby incorporated by reference.

### BACKGROUND

1. Field

The present disclosure relates to a method of screening a protein capable of binding to a compound.

2. Description of the Related Art

Systems biology is a field of study that focuses on the expression and interaction of genes, proteins, and metabolites to help the understanding of genome and metabolite pathways in a systematical manner.

Several approaches to identify a protein binding to a specific compound are available in the art. However, in these approaches, non-specific binding of proteins to target compounds limits the ability to isolate a highly reliable target-binding protein.

Therefore, methods of screening a protein binding to a compound in a more efficiently manner are required.

### SUMMARY

Provided is a method of screening for a protein that binds to a target compound. The method comprises receiving data describing two or more candidate proteins expected to bind to a target compound; obtaining a gene ontology (GO) term for each of the candidate proteins from a GO database containing information about biological properties of the two or more candidate proteins; obtaining two or more genes that show a statistically significant change in expression when the target compound is in contact with a cell from a gene expression database; obtaining enriched GO terms for the two or more genes, which are GO terms that are repeated among GO terms for the two or more genes from a GO database containing information about biological properties of the two or more genes; and selecting a protein from among the two or more candidate proteins that has a GO term identical to the enriched GO term by comparing the enriched GO term with the GO term for each of the two or more candidate proteins, wherein each step of the method is performed by one or more processors.

Additional aspects will be set forth in part in the description which follows and, in part, will be apparent from the description, or may be learned by practice of the presented exemplary embodiments.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1A is a schematic diagram of a method of screening for a protein that binds a target compound;

FIG. 1B is a block level diagram of a device for screening for a protein that binds a target compound; and

FIG. 2 shows GO identifiers enriched for differentially expressed genes (DEG-enriched GO) related to dasatinib treatment.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed items.

In one embodiment, a method of screening for a protein that binds to a specific target compound includes: identifying, obtaining, and/or receiving data describing two or more candidate proteins expected to bind to a specific target compound; identifying and/or obtaining a relevant gene ontology (GO) term for each of the proteins from a GO database containing information about biological properties of the two or more proteins; indentifying, obtaining, and/or receiving data describing two or more genes that show a statistically significant change in expression when the target compound is in contact with a cell (Differentially Expressed Genes or “DEGs”) from a gene expression database; obtaining enriched GO terms for the two or more genes from a GO database containing information about biological properties of the two or more genes; and selecting a protein that contains a GO term identical to an enriched GO term by comparing the enriched GO terms with the GO terms of each of the candidate proteins. Each step of the method may be performed by one or more processors.

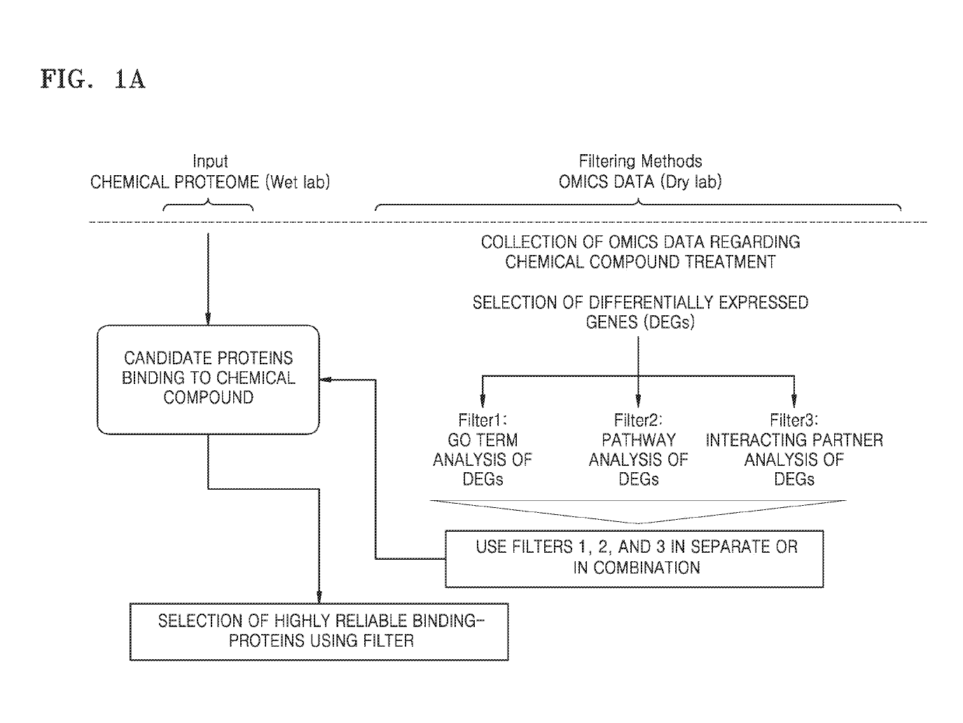

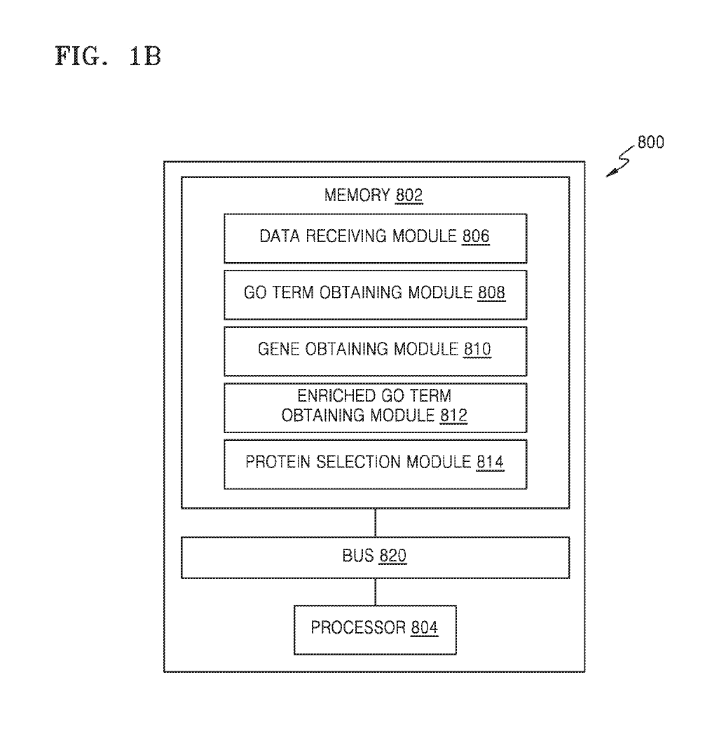

The method may be performed by the device as shown in FIG. 1B. The device (800) may include processor(s) (804), and memory (802) coupled to the processor(s) (804). The memory (802) includes a plurality of modules stored in the form of executable program code which instructs the processor(s) (804) to perform each step in the method. The memory (802) may include data receiving module (806), GO term obtaining module (808), gene obtaining module (810), enriched GO term obtaining module (812), and protein selection module (814).

The data receiving module (806) may instruct the processor(s) (804) to receive data describing two or more candidate proteins expected to bind to a target compound. The GO term obtaining module (808) may instruct the processor(s) (804) to obtain a gene ontology (GO) term for each of the candidate proteins from a GO database containing information about biological properties of the two or more candidate proteins. The gene obtaining module (810) may instruct the processor(s) (804) to obtain two or more genes that show a statistically significant change in expression when the target compound is in contact with a cell from a gene expression database. The enriched GO term obtaining module (812) may instruct the processor(s) (804) to obtain enriched GO terms for the two or more genes, which are GO terms that are repeated among GO terms for the two or more genes from a GO database containing information about biological properties of the two or more genes. The protein selection module (814) may instruct the processor(s) (804) to select a protein from among the two or more candidate proteins that has a GO term identical to the enriched GO term by comparing the enriched GO term with the GO term for each of the two or more candidate proteins.

The candidate proteins may be obtained from data resulting from in vitro, in vivo, or in situ experiments (e.g., “wet” chemical experiments). The data may be publicly available or not yet published. The candidate proteins may be expected to bind to the target protein, for example, based on the result of a pull-down assay, in which a candidate protein is “pulled” out of solution due to the interaction between a protein and a target compound (e.g., precipitated, bound to a immobilized reagent, etc.). For instance, the candidate protein may be captured by a probe including a reactive group of the target compound. The candidate protein may be present in a cellular lysate or a live-cell. Alternatively, or in addition, the candidate protein may be identifyed according to a cell-based proteome profiling method or a chemical proteomics method. Proteins identified as candidate proteins can be input into the data receiving module (806).

The term “gene ontology (GO)” used herein refers to information about biological properties of genes and gene products in terms of a cellular component, a molecular function, or a biological process. The cellular component information refers to location of a gene or a protein within a cell. The molecular function information refers to function of a protein at the molecular level. The biological process information refers to roles of a biological process. A “GO term” is a term that reflects one or more such biological properties. GO terms are known in the art (see, e.g., The Gene Ontology Consortium, (2015) Nucl Acids Res, 43, Database issue D1049-D1056, available at <www.geneontolgy.org>>; “Gene Ontology: tool for the unification of biology” (2000) Nature Genet, 25 (1): 25-9).

The GO database may contain information, in the form of GO terms, about a biological property of a gene or a gene product. The information about the biological property may include cellular component information, molecular function information, and/or biological process information of the gene. The GO database may provide, in response to an input of data identifying or describing a gene, a relevant GO term for biological properties of the gene or the gene product (e.g., protein). The input of the gene may be a gene symbol or a gene identification (ID) number. The GO term may be classified or identified according to a GO ID, a GO name, a GO definition, or a combination thereof. GO databases are publically available (e.g., The Gene Ontology Consortium, (2015) Nucl Acids Res, 43, Database issue D1049-D1056, available at <<www.geneontolgy.org>>; “Gene Ontology: tool for the unification of biology” (2000) Nature Genet, 25 (1): 25-9).

A gene expression database is a database that contains information about a change in gene expression levels resulting from treatment with the specific target compound. The change in the gene expression level may be a change at a transcript level, a protein level, or a combination thereof. The gene expression database may be a microarray database. The gene expression database may include information about the transcript expression level in a cell in contact with the compound compared to a cell not in contact with the compound. The cell may be derived from a human, a mouse, a cow, a pig, a horse, a bacterium, a yeast, or a drosophila. Gene expression databases suitable for use with the method include, for instance, Gene Expression Omnibus database (GEO; <<www.ncbi.nlm.nih.gov/geo/>>), Stanford microarray database.

An enriched GO term is a term that repeats in the GO terms of the two or more genes among genes that are differentially expressed in a cell when contacted with the target compound. Thus, for instance, a set of differentially expressed genes corresponding to treatment with a given compound will have a set of GO terms associated with the gene set. The enriched GO terms are those terms that appear in the GO term set more than once (e.g., at least 2, 3, 4, 5, or 6 times or more).

A candidate protein is selected according to the screening method if it has one or more GO terms that matches one or more enriched GO terms of the DEGs. Thus, the method can include comparing the enriched GO terms with the relevant GO terms of the candidate proteins. A protein containing one or more GO terms that are the same as one or more enriched GO terms (e.g., at least 2, 3, 4, or 5 GO terms identical to the enriched GO terms) may be selected as a target compound-specific binding protein.

Hereinafter, the present invention will be described in further detail with reference to the following examples. These examples are for illustrative purposes only and are not intended to limit the scope of the present invention.

### Example 1

### Selection of Dasatinib-Binding Protein

1.1. Information Obtained about Candidate Dasatinib-Binding Proteins

A list of 239 candidate proteins, which were selected as dasatinib-binding proteins based on cell-based proteome profiling using an affinity-based probe (Haibin et al., JACS, 2012, 134:3001-3014), was obtained. According to several related arts (Nat. Chem. Biol (2010), 6, 291; PNAS (2007), 104, 13283; JACS (2012), 134, 3001), it was confirmed whether each of these candidate proteins actually binds to dasatinib, and as a result, the total candidate proteins on the list were divided into 22 dasatinib-binding proteins and 217 false-positive binding proteins. The gene names of 22 dasatinib-binding proteins are SRC, HCK, PRKCB, EGFR, CSK, AURKA, AURKAPSI, Cdk18, EIF2AK2, LYN, MAPK14, BTK, YES1, PRKDC, LOC731751, TWF1, PRKACA, STAT3, STK25, CDK2, PKN2, and FYN.

1.2. Manufacture of Filter Using Omics Data

Microarray data showing gene expression changes in a K562 cell (human myeloid leukemia cell line) treated with Dasatinib was collected from the Gene Express Omnibus (GEO) database (GEO accession: GSE51083). GEO is the public repository for high throughput data at NCBI. GEO contains microarray and other transcriptome data in MIAME compliant formats and ChIP-chip data. GEO can be searched from either the GEO search page (http://www.ncbi.nlm.nih.gov/geo/) or from the Entrez home page. The collected data was statistically analyzed to select differentially expressed genes (DEGs) whose expression significantly increased or decreased depending on the dasatinib treatment. Here, genes satisfying all conditions including: absolute value (fold change)>=0.58 (log 2 scale) (condition 1); p-value of t-test<0.01 (condition 2); and the minimum expression value of a control group and an experimental group is equal to or greater than 7 (condition 3), were selected as DEGs for dasatinib.

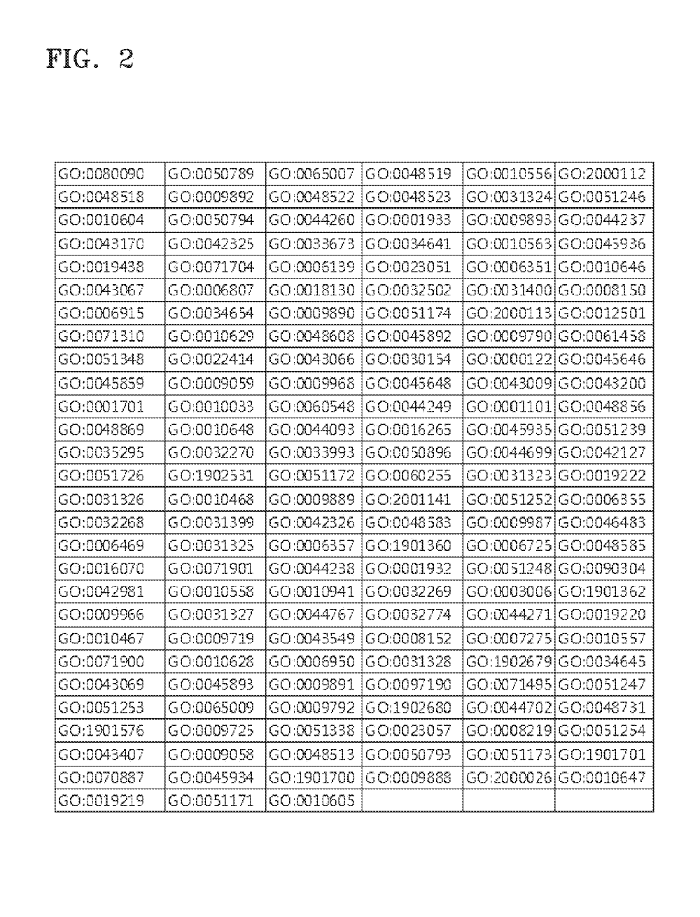

Afterwards, DEG-enriched GO concentrated in the selected DEGs were analyzed using a publically available GO database. In detail, EntrezlD or symbol list of the selected DEGs was input into an enrichment analysis window on the internet site (http://geneontology.org/), “biological process” and “H. sapiens” were checked, and all the inputs were submitted. FIG. 2 shows identifiers of the DEG-enriched GO for dasatinib.

1.3. Filtration of Candidate Dasatinib-Binding Proteins

The relevant GO terms to each of the candidate dasatinib-binding proteins obtained according to Example 1.1 were mapped based on the GO database. For each of the candidate dasatinib-binding proteins, the number of DEG-enriched GO terms regarding dasatinib obtained according to Example 1.2 that matched the GO terms of the candidate protein was determined, and then, the actual binding capability to dasatinib was determined as either ‘positive’ or ‘negative’. For example, LYN was identified as a candidate protein in Example 1.1. Among the GO terms mapped regarding an LYN gene, six of the mapped GO terms matched the DEG-enriched GO terms for dasatinib. In addition, since the protein expressed by the LYN gene were confirmed to actually bind to dasatinib, the LYN gene was noted ‘positive’. Genes were sorted in a descending order of genes having more GO terms matched to DEG-enriched GO terms for dasatinib. Here, the cut-off value of the sorting was set to be 3. Genes passing the cut-off value that were also confirmed to actually bind to dasatinib included LYN, MAPK14, STAT3, BTK, PKN2, and PRKCB genes.

Table 1 below shows the number of genes that were confirmed to bind or not to bind to dasatinib according to experiments, and the number of genes that are expected to bind or not to bind to dasatinib according to the filtration.

TABLE 1

Experimental results

| | | Positive | Negative |
|---|---|---|---|
| Filtration results | Positive | 6 | 25 |
| | Negative | 16 | 192 |

According to the filtration, it was concluded that 6 out of 22 proteins binding to dasatinib were expected as dasatinib-binding proteins. In addition, 192 out of 217 proteins not binding to dasatinib were expected as dasatinib-non-binding proteins. Therefore, it was confirmed that the filtration was capable of effectively excluding false-positive proteins.

As described above, according to the one or more of the above exemplary embodiments, a highly reliable binding protein with respect to a specific compound may be screened.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. A method of screening for a protein that binds to a target compound, the method comprising:
receiving data describing two or more candidate proteins expected to bind to a target compound;
obtaining a gene ontology (GO) term for each of the candidate proteins from a GO database containing information about biological properties of the two or more candidate proteins;
obtaining two or more genes that show a statistically significant change in expression when the target compound is in contact with a cell from a gene expression database;
obtaining enriched GO terms for the two or more genes, which are GO terms that are repeated among GO terms for the two or more genes from a GO database containing information about biological properties of the two or more genes; and
selecting a protein from among the two or more candidate proteins that has a GO term identical to the enriched GO term by comparing the enriched GO term with the GO term for each of the two or more candidate proteins,
wherein each step of the method is performed by one or more processors.

2. The method of claim 1, wherein the two or more candidate proteins are expected to bind to the target compound by a pull-down assay.

3. The method of claim 2, wherein the two or more candidate proteins are captured by a probe comprising a reactive group of the target compound.

4. The method of claim 2, wherein the two or more candidate proteins are present in a cellular lysate or a live-cell.

5. The method of claim 1, wherein the two or more candidate proteins are expected to bind to the target compound by cell-based proteome profiling method or chemical proteomics method.

6. The method of claim 1, wherein the information about the biological properties comprises cellular component information, molecular function information, biological process information, or a combination thereof.

7. The method of claim 1, wherein the GO database provides, in response to an input of data describing a gene or protein, a relevant GO term for biological properties of the gene or protein.

8. The method of claim 7, wherein the input of the gene or protein is input of a gene or protein symbol or a gene identification (ID) number.

9. The method of claim 1, wherein the GO term is identified according to a GO ID, a GO name, a GO definition, or a combination thereof.

10. The method of claim 1, wherein the two or more genes that that show a statistically significant change in expression when the target compound is in contact with a cell are identified from a gene expression database that contains information about a change in gene expression level according to a treatment with the target compound.

11. The method of claim 10, wherein the change in gene expression level comprises a change at a transcript level, a protein level, or a combination thereof.

12. The method of claim 1, wherein the gene expression database contains information about the transcript expression level in a cell in contact with the target compound compared to a cell not in contact with the compound.

13. The method of claim 1, wherein the protein selected from the two or more candidate proteins has at least 2 GO terms identical to the enriched GO terms.
